(19) United States
(12) Patent Application Publication
(10) Pub. No.: US 2024/0148424 A1
(43) Pub. Date: May 9, 2024

(54) APPARATUS AND METHOD FOR MARGINAL ABLATION IN TISSUE CAVITY

(71) Applicant: SenoGuard, Inc., Seal Beach, CA (US)

(72) Inventors: Greig Eric Altieri, Charlotte, NC (US); Diana Lee Tucker, Seal Beach, CA (US); Roberta Lee, Seal Beach, CA (US); Darius S. Francescatti, Barrington, IL (US); Sean Carroll, Pasadena, CA (US)

(73) Assignee: SenoGuard, Inc., Seal Beach, CA (US)

(21) Appl. No.: 18/548,064

(22) PCT Filed: Feb. 28, 2022
(86) PCT No.: PCT/US2022/018180

§ 371 Date: Aug. 25, 2023

(51) Int. Cl. A61B 18/02; A61B 18/00

(52) U.S. Cl. CPC A61B 18/02; A61B 2018/00095; A61B 2018/00333; A61B 2018/00577; A61B 2018/00744; A61B 2018/00797; A61B 2018/0262; A61B 2018/0293

(57) ABSTRACT

A probe for ablating a marginal tissue region in a surgically created tissue cavity includes a shaft having a shell with an exterior heat transfer surface mounted on a distal region of the shaft. At least one temperature sensor is provided on the exterior heat transfer surface of the shell, and the exterior heat transfer surface contacts at least a portion of an inner surface of the surgically created tissue cavity when placed therein. A cryogenic system supplies cryogenic fluid to a supply lumen and removes the fluid through separate fluid removal lumen in the shaft.

### CROSS-REFERENCE TO RELATED APPLICATIONS

This application claims the benefit of U.S. Provisional No. 63/154,561, (Attorney Docket No. 57648-704.101), filed Feb. 26, 2021, the entire content of which is incorporated herein by reference.

### BACKGROUND OF THE INVENTION

### 1. Field of the Invention

This invention relates generally to medical devices and methods. More particularly, this invention relates to methods and devices for treating a margin of soft tissue within the walls of a surgical cavity in a breast following the removal of a cancerous tumor.

The current standard of care for breast conservation is irradiation of the whole breast or a segment of the breast to eradicate any residual cancer cells following a lumpectomy or excision of a cancer. As an alternative to mastectomy, breast conservation is the recommended local treatment in most breast cancers, however radiation therapy has serious shortcomings for the patient both in the gross overtreatment of tissue as well as potential future significant complications. Short term complications include skin burns, woody breast texture, tissue distortion and shrinkage. Long term complications include lymphedema of the arm, delayed cardiotoxicity, and, rarely, the development of a secondary non breast cancer.

Newer technologies have allowed physicians a more targeted approach to reducing the incidence of future cancers. The aim of these technologies is to destroy tissues beyond the surgical cavity walls to a depth of between 5 and 20 mm. One method utilizes brachytherapy tools to place a radiation source within the surgical cavity at the time of the operation to deliver a focused radiation therapy treatment. While this technology has proven to be both clinically effective and convenient for the patient, it still carries the risks of both short and long-term effects of radiation exposure and is especially costly to the healthcare system. Other technologies are in a more experimental state and have included intracavitary radio-frequency ablation to treat the cavity margin, or the use of other forms of energy to treat the walls of the surgical cavity. These energy forms have included high intensity focused ultrasound, laser, microwave, and cryoablation.

### 2. Background Art

US 2021/0153920 has common inventorship herewith, the full disclosure of which is incorporated herein by reference. Cryogenic balloons are described in, for example, U.S. Pat. Nos. 9,603,650; 9,414,878; 9,402,676; and 7,740,627.

### SUMMARY OF THE INVENTION

A fixed probe system for supplying cryogenic energy to the surgical cavity is described herein. The solid fixed probe, constructed of materials typically used in medical devices such as stainless steel, cobalt-chrome alloy, titanium, and nickel-titanium alloy, is inserted into the surgical cavity through a previously created surgical incision or tract used to excise the cancer or through a newly created tract. Following placement, a selected cryogenic agent is administered through a channel into one or more discrete channels affixed to the surface of the probe for supplying cryogenic energy to the surface of the fixed probe and removing energy from the surrounding tissue. Preferably the probe is sized or configured for maximum contact with the walls of the surgical cavity.

The fixed probe system is coupled to a cryogenic fluid delivery system with one or more distal outflow port(s) and one or more inflow port(s) for supplying cooling energy to the exterior of the fixed probe and removing the latent heat of the surrounding tissue to cause necrosis of the surrounding tissue. The cryogenic fluid delivery system may be as simple as a handheld fluid delivery device or as complicated as a microprocessor controlled closed loop fluid delivery system depending on the selected cryogenic fluid. Typically, the cooling system will comprise a Joule-Thompson effect cooler or other system that relies on expansion and phase change of a liquid passing through a valve. In other examples, the cooling system may employ evaporative cooling, e.g., using a state point liquid cooling (SPLC) system.

Another embodiment of the fixed probe system for supplying cryogenic energy to the breast tumor cavity has a fixed probe constructed of a hollow member, either spherical or ellipsoidal. The interior of the hollow member may be at atmospheric pressure or at a vacuum to assist in insulating the interior of the fixed probe to maximize thermal energy transfer to the surrounding tissue.

Another embodiment of the fixed probe system for supplying cryogenic energy to the breast tumor cavity has a fixed probe constructed of a mesh member either spherical or ellipsoidal, with one or more discrete channels affixed to the surface of the probe for supplying cryogenic energy to the surface of the fixed probe and removing energy from the surrounding tissue.

Another embodiment of the fixed probe system for supplying cryogenic energy to the breast tumor cavity optionally incorporates a suction path between exterior of the tissue contacting exterior surface fixed probe element and the tissue cavity. This suction path can be added to any of the above fixed probe configurations to eliminate any gap, whether air or fluid, between the tissue contacting fixed probe and tissue to assure complete geographic delivery of cryogenic energy to the targeted tissue.

Another embodiment of the fixed probe system for supplying cryogenic energy to the breast tumor cavity optionally incorporates a system of thermal measurement to monitor the progression of isotherms from the probe surface into the tissue and give feedback to the system allowing for control of the isotherm progression to adhere to a specified treatment regimen.

### BRIEF DESCRIPTION OF THE DRAWINGS

FIGS. 1A-1E, taken from US 2021/0153920 previously incorporated herein by reference, illustrate in cross-section a breast and the steps for treatment of a breast cancer using a cryogenic probe system.

FIG. 2 illustrates in a cross-sectional view a single fixed probe system constructed in accordance with the principles of the present invention for use with a cryogenic fluid that provides the delivery of a cryogenic fluid to the surgical cavity within the breast.

FIG. 3 illustrates in a cross-sectional view a single fixed probe system constructed in accordance with the principles of the present invention for use with a cryogenic fluid that provides the delivery of a cryogenic fluid to the surgical cavity within the breast using a simplified coaxial inflow and outflow method.

FIG. 4 illustrates in cross-sectional view a signal fixed probe system constructed in accordance with the principles of the present invention for use with a cryogenic fluid that provides the delivery of a cooling agent to the surgical cavity within the breast with a means of directly cooling the entire fixed probe.

FIG. 5 illustrates in a cross-sectional view a single fixed probe system constructed in accordance with the principles of the present invention for use with a cryogenic fluid that provides the delivery of a cryogenic fluid to the surgical cavity within the breast and provides a suction path exterior to the tissue contacting fixed probe.

FIG. 6 illustrates two orthogonal views of a signal fixed probe system constructed in accordance with the principles of the present invention for use with a cryogenic fluid that provides the delivery of a cooling agent to the surgical cavity within the breast with a means of directly cooling the entire fixed probe.

FIG. 7 illustrates two orthogonal views of the signal fixed probe system of FIG. 6 with a temperature sensor for monitoring the progression of isotherms from the surface of the probe through the tissue so that the depth of tissue cooled below a selected lethal isotherm can be determined.

### DETAILED DESCRIPTION OF THE INVENTION

Fixed probe systems to deliver cryogenic cooling to the walls of a surgical cavity are described. In one embodiment, a fixed probe delivers cryogenic energy to lethally freeze a margin of soft tissue surrounding a surgical cavity in the breast to ablate or necrotize any remaining cancerous or precancerous cells. Optionally, an additional configuration includes a secondary suction channel to improve tissue contact with the fixed probe to increase system efficiency and reduce procedure time. The cryogenic fluid agents that are utilized are particularly useful for rapidly ablating or necrotizing soft tissue walls of a surgical cavity. Any number of cryogenic fluids may be utilized in the fixed probe system, including but not limited to, nitrous oxide, liquid nitrogen, super critical nitrogen, helium, oxygen or argon.

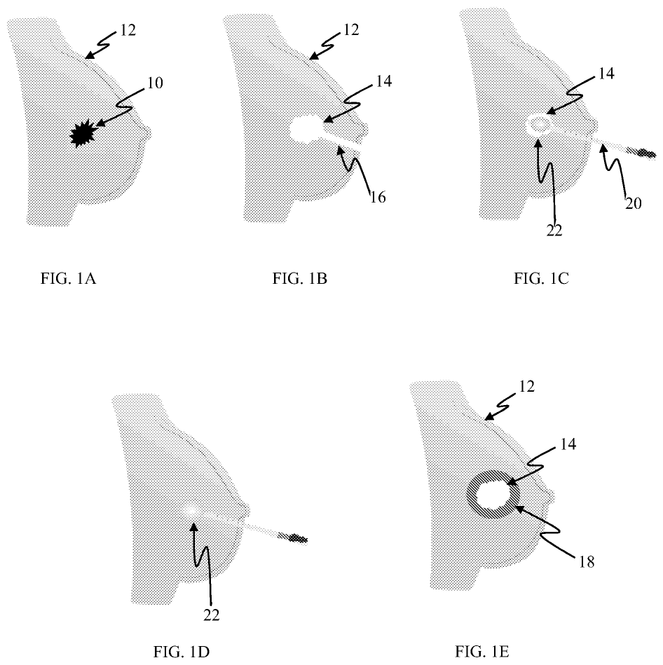

FIG. 1 illustrates in cross-section a breast at steps of treatment for the surgical excision of a breast cancer followed by fixed probe cryo-ablation of a margin of soft tissue in the walls of the surgical cavity. FIG. 1A illustrates a cancer 10 in the breast 12. FIG. 1B illustrates a surgical cavity 14 in breast 12 with a surgical tract 16 after the cancer has been excised. Surgical excision may be performed using open surgical techniques or using one or more devices that incorporate minimally invasive access. FIG. 1E illustrates fixed probe system 20 placed within the surgical tract with the fixed probe 22 in surgical cavity 14. FIG. 1D illustrates the fixed probe 22 with cryogenic fluid and commencement of ablation of the margin of soft tissue in the walls of the surgical cavity. FIG. 1E illustrates the breast 12 after the fixed probe system has been removed and the surgical tract closed. A margin of soft tissue 18 within the wall of the surgical cavity 14 has been ablated.

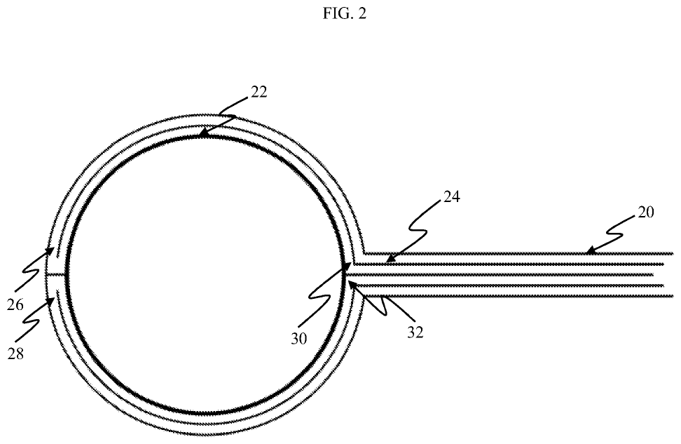

FIG. 2 illustrates a cross-sectional view of a fixed probe cryo-ablation device 20. The tissue contacting fixed probe 22 is supported by at least one co-axial tube(s) 24 on the exterior of the fixed probed or imbedded into the surface of the fixed probe. Once the fixed probe cryoablation device 20 is inserted into the breast, cryogenic fluid is infused into and circulated along the exterior of the tissue contacting fixed probe 22 entering through supply tube 26 and, optionally a second supply tube 28, and exiting the tissue contacting fixed probe 22 through cryogenic fluid return tube 30 and optionally a second return tube 32.

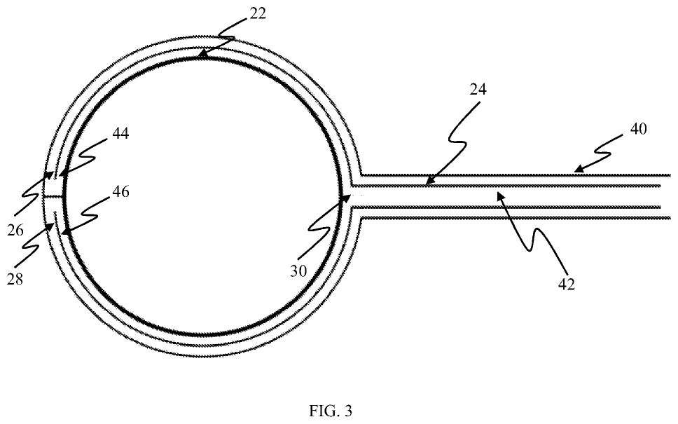

FIG. 3 illustrates a cross-sectional view of a fixed probe cryo-ablation device 40 with a unified cryogenic fluid supply or return fluid path. Once the fixed probe cryo-ablation device 40 is inserted into the breast, cryogenic fluid is infused into and circulated along the exterior of the tissue contacting fixed probe 22 entering through supply tube 26 and, optionally a second supply tube 28, and exiting the tissue contacting fixed probe 22 through a single cryogenic fluid return tube 30. The flow direction of the cryogenic fluid can alternatively be reversed entering through supply tube 42, and exiting the tissue contacting fixed probe 22 through cryogenic fluid return tube 44 and, optionally a secondary fluid return tube 46.

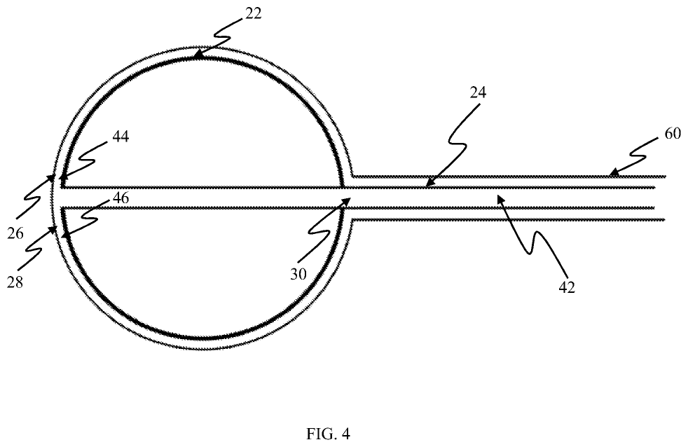

FIG. 4 illustrates a cross-sectional view of a fixed probe cryo-ablation device 60 with a unified cryogenic fluid supply or return fluid path through the center of the tissue contacting fixed probe 22. Once the fixed probe cryo-ablation device 60 is inserted into the breast, cryogenic fluid is infused into and circulated along the exterior of the tissue contacting fixed probe 22 entering through supply tube 26 and, optionally a second supply tube 28, and exiting the tissue contacting fixed probe 22 through a single cryogenic fluid return tube 30 through the center of the tissue contacting fixed probe 22. The flow direction of the cryogenic fluid can alternatively be reversed entering through supply tube 42, and exiting the tissue contacting fixed probe 22 through cryogenic fluid return tube 44 and, optionally a secondary fluid return tube 46.

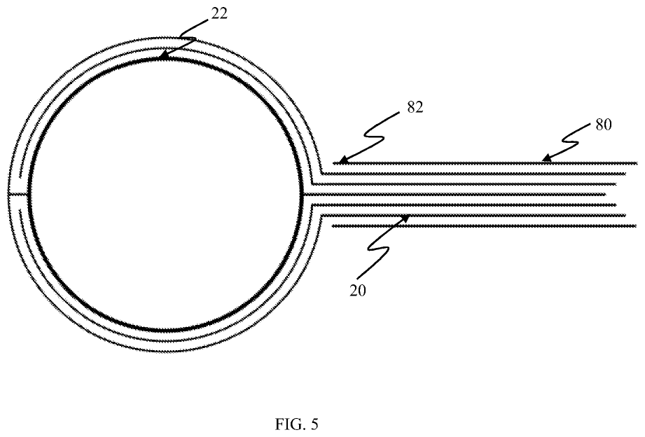

FIG. 5 illustrates a cross-sectional view of a single fixed probe cryo-ablation device 80 with the additional of a co-axial suction channel 82 around the periphery of the proximal portion of a fixed probe cryo-ablation device 20 to remove excess air or fluid between the tissue contacting fixed probe 22 and the tissue cavity. Once this material has been removed, the cryo-ablation process can be initiated.

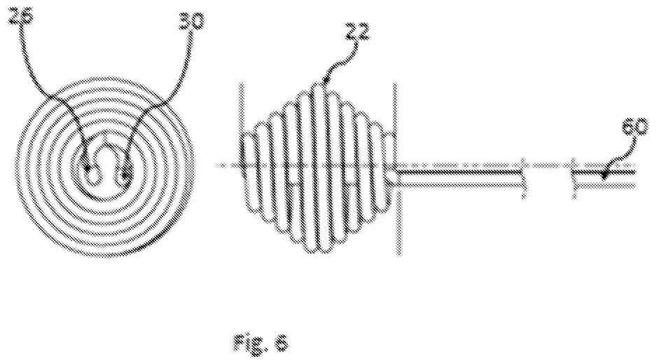

FIG. 6 illustrates two orthogonal views of an alternate fixed probe cryo-ablation device 60 with a unified cryogenic fluid supply or return fluid path through the length of the tissue contacting fixed probe 22. Once the fixed probe cryo-ablation device 60 is inserted into the breast, cryogenic fluid is infused into and circulated along the interior of the tissue contacting fixed probe 22 entering through supply tube 26 and exiting the tissue contacting fixed probe 22 through a single cryogenic fluid return tube 30 through the center of the tissue contacting fixed probe 22.

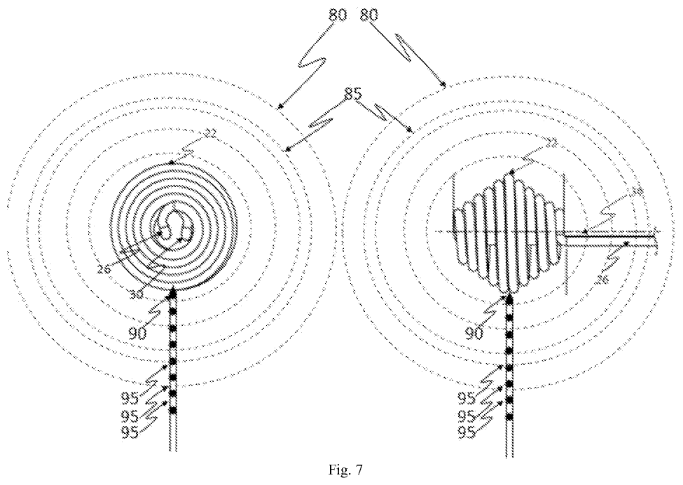

FIG. 7 illustrates the system for isotherm monitoring from the surface of the fixed probe cryoablation device 22. The temperature sensing probe 90 is inserted until it makes contact with the cryoablation device 22. Once both are in place in the breast, the cryogenic fluid is circulated through the supply lumen 26 and exits through the return lumen 30. The temperature at the surface of the device is monitored by the temperature sensor at the tip of the probe 90 and the progression of the isotherms through the tissue is measured by multiple thermal sensors 95 located along the length of the probe 90. The leading isotherm 80 will be warmer than all the trailing isotherms and the lethal isotherm 85 will be selected based on the type of tumor being treated. Isotherm progression can be controlled by direct feedback from the various temperature probes 95 to the system for controlling the flow and temperature of the cryogenic fluid. Any temperature monitoring system may be employed to measure the progression of the isotherms. Examples of other temperature monitoring systems are infrared, microwave, MRI and ultrasound.

Any elements described herein as singular can be pluralized (i.e., anything described as “one” can be more than one). Any species element of a genus element can have the characteristics or elements of any other species element of that genus. The media delivered herein can be any of the fluids (e.g., liquid, gas, or combinations thereof) described herein. The patents and patent applications cited herein are all incorporated by reference herein in their entireties. Some elements may be absent from individual figures for reasons of illustrative clarity. The above-described configurations, elements or complete assemblies and methods and their elements for carrying out the disclosure, and variations of aspects of the disclosure can be combined and modified with each other in any combination. All devices, apparatuses, systems, and methods described herein can be used for medical (e.g., diagnostic, therapeutic or rehabilitative) or non-medical purposes.

1-20. (canceled)

21. A method for removing a tumor from tissue, said method comprising:
surgically removing the tumor from the tissue to create a tissue cavity having an exposed tissue surface;
inserting a shell having an outer heat transfer surface surrounding an interior volume into the tissue cavity, wherein at least a portion of the outer surface engages the exposed tissue surface; and
circulating a cooling fluid through the interior volume (i) under conditions selected to cool and ablate the exposed tissue surface to a predetermined end point or (ii) in order to cool and ablate the tissue and controlling the depth of tissue ablation by monitoring the progression of isotherms through the tissue.

22. The method as in claim 21, wherein the shell has at least two generally concentric walls defining the interior volume that receives the circulating cooling fluid.

23. The method as in claim 22, wherein the interior volume between the at least two generally concentric walls is divided into at least two isolated interior regions, each of which regions receives a separate flow of the cooling fluid.

24. The method as in claim 23, wherein each of the separate flows of the cooling fluid can be independently controlled.

25. The method as in claim 21, wherein the shell has a generally spheroidal or ovoidal shape.

26. The method as in claim 21, further comprising cooling the cooling fluid prior to circulating the cooling fluid through the interior volume.

27. The method as in claim 21, wherein the cooling fluid cools as it is released into the interior volume.

28. The method as in claim 21, wherein the cooling fluid undergoes a Joule Thomson expansion to lower its temperature.

29. The method as in claim 21, wherein circulating the cooling fluid comprises delivering the fluid to the shell and collecting the cooling fluid from the shell through coaxial lumens in a shaft attached to a probe.

30. The method as in claim 21, further comprising stopping circulation of the cooling fluid through the interior volume of the expanded surface, circulating a warming fluid through the interior volume of the expanded surface, and circulating a cooling fluid the interior volume of the expanded surface a second time.

31. The method as in claim 30, further comprising controlling circulation of the cooling and warming fluids through the interior volume of the expanded surface, such that the temperature of the probe is programmable by the user to optimize the treatment.

32. The method as in claim 30, further comprising controlling circulation of the cooling and warming fluids through the interior volume of the expanded surface, such that the total heat transfer capacity of the probe is programmable by the user to optimize the treatment.

33. The method as in claim 21, wherein the end point is any one or more of a predetermined ablation depth, a predetermined tissue temperature, a predetermined treatment duration, and a predetermined cooling profile.

34. A probe and system for ablating a marginal tissue region in a surgically created tissue cavity, said probe and system comprising:
a shaft;
a shell mounted on a distal region of the shaft having an exterior heat transfer surface with temperature sensors surrounding an interior volume at a distal end of the shaft, said exterior heat transfer surface configured to contact at least a portion of an inner surface of the surgically created tissue cavity when placed therein; and
a cryogenic system to supply fluid to a supply lumen and a separate cryogenic fluid removal lumen, both lumens being disposed in the shaft and connected to the system.

35. The probe of claim 34, wherein the cryogenic fluid supply lumen is disposed concentrically about the cryogenic removal lumen.

36. The probe of claim 34, wherein the shaft further comprises a secondary fluid supply lumen.

37. The probe of claim 34, wherein the shell has at least two generally concentric walls defining the interior volume that receives the circulating cooling fluid.

38. The probe of claim 37, wherein the interior volume between the at least two generally concentric walls is divided into at least two isolated interior regions, each of which regions receives a separate flow of the cooling fluid.

39. The probe of claim 38, wherein each of the separate flows of the fluid can be independently controlled.

40. The probe of claim 34, wherein the shell has a generally spheroidal or ovoidal shape.

41. The probe of claim 34, wherein the fluid is cooled prior to circulating the cooling fluid through the interior volume.

42. The probe of claim 34, wherein the fluid cools as it is released into the interior volume.

43. The probe of claim 34, wherein the fluid is cooled by undergoing a Joule Thomson expansion to lower its temperature.

44. The probe of claim 34, wherein the cryogenic system is configured to stop circulation of a first cooling fluid through the interior volume of the expanded surface, circulate a warming fluid through the interior volume of the expanded surface, and circulate a second cooling fluid the interior volume of the expanded surface a second time.

45. The probe of claim 44, wherein the cryogenic system is further configured to control circulation of the cooling and warming fluids through the interior volume of the expanded surface, such that the temperature of the probe is programmable by the user to optimize the treatment.

46. The probe of claim 44, wherein the cryogenic system is further configured to control circulation of the cooling and warming fluids through the interior volume of the expanded surface, such that the total heat transfer capacity of the probe is programmable by the user.

47. The probe of claim 34, wherein the cryogenic system is configured to circulate a cooling fluid through the interior volume under conditions selected to cool and ablate the exposed tissue surface to one or more predetermined end points, wherein the end points include any one or more of a predetermined ablation depth, a predetermined tissue temperature, a predetermined treatment duration, and a predetermined cooling profile.
